(19) United States
(12) Patent Application Publication
(10) Pub. No.: US 2018/0369508 A1
(43) Pub. Date: Dec. 27, 2018

(54) AIR-FLOW IN A NEBULIZER HEAD

(71) Applicant: KONINKLIJKE PHILIPS N.V., EINDHOVEN (NL)

(72) Inventors: DIRK ERNEST VoON HOLLEN, CLARK, NJ (US); FILIPPO QUADRELLI, RUSTINGTON (GB); MICHAEL DUNCAN, PORTSMOUTH (GB)

(21) Appl. No.: 16/064,497

(22) PCT Filed: Dec. 21, 2016
(86) PCT No.: PCT/IB2016/057847

§ 371 Date: Jun. 21, 2018

Related U.S. Application Data
(60) Provisional application No. 62/271,387, filed on Dec. 28, 2015.

(51) Int. Cl. A61M 11/00

(52) U.S. Cl. CPC A61M 11/002; A61M 15/0001; A61M 11/006

(57) ABSTRACT

The present invention relates to air-flow in a nebulizer head. In order to provide a nebulizer head with an improved air-flow guidance, a nebulizer head (10) comprises a housing structure (12) providing an air-flow path (16) with two opposite air-flow openings (18a, 18b), and a fluid reservoir (14) provided to accommodate a liquid from which small droplets are to be generated in order to form an aerosol, and an aerosol generator (20) with an aperture member (22) that comprises an aperture surface (24) with a plurality of apertures to provide the small droplets from the liquid. The fluid reservoir is arranged adjacent the aerosol generator such that the aperture member is in contact with the fluid. The aerosol generator is arranged within the air-flow path. At least one by-pass flow path (26) is provided between the aerosol generator and the housing structure. The air-flow path is a bidirectional path that provides a first air-flow direction (28) for inhaling, and a second air-flow direction (38) for exhaling. The air enters the air-flow path and leaves the air-flow path via the air-flow openings. The aperture surface is arranged transverse to the first and second air-flow direction.

### FIELD OF THE INVENTION

The present invention relates to air-flow in a nebulizer head, and relates in particular to a nebulizer head, to a nebulizer system and to a method for providing a substance in an aerosolized form using a nebulizer.

### BACKGROUND OF THE INVENTION

For inhalation purposes, for example for inhalation therapy of applying a drug to a patient, a liquid may be used to generate a plurality of small droplets, which is also called atomization of the liquid, and to generate a mist or aerosol that can be inhaled by the patient. For the atomization of the liquid, compressed air, ultrasonic or mesh droplet generators are provided, as examples. In US 2008/0000470 A1, an inhalation therapy device is provided in which an air-flow is provided by air conveying means and liquid droplets and the air-flow are mixed in a nebulizing chamber to provide the mixture to be inhaled. After inhaling, i.e. during exhaling, aerosol droplets may still be present in the exhaled air stream. However, it has been shown that when discharging the exhaled air droplet mixture into the environment, at least for certain drug medication, care must be taken that the drug medication does not get on the face of the patient, for example into the eyes.

### SUMMARY OF THE INVENTION

There may thus be a need to provide a nebulizer head with an improved air-flow guidance.

The object of the present invention is solved by the subject-matter of the independent claims, wherein further embodiments are incorporated in the dependent claims.

According to the present invention, a nebulizer head is provided comprising a housing structure, a fluid reservoir and an aerosol generator. The housing structure provides an air-flow path with two opposite air-flow openings. The fluid reservoir is provided to accommodate a liquid from which small droplets are to be generated in order to form an aerosol. The aerosol generator has an aperture member that comprises an aperture surface with a plurality of apertures to provide the small droplets from the liquid. The fluid reservoir is arranged adjacent the aerosol generator such that the aperture member is in contact with the fluid. The aerosol generator is arranged within the air-flow path. At least one by-pass flow path is provided between the aerosol generator and the housing structure. The air-flow path is a bi-directional path that provides a first air-flow direction for inhaling and a second air-flow direction for exhaling. The air enters the air-flow path and leaves the air-flow path via the air-flow openings. The aperture surface is arranged transverse to the first and second air-flow direction.

Due to arranging the air-flow path having at least one by-pass flow path around the aerosol generator, which is arranged between the two openings, the air exits at the rear side, i.e. opposite to the face side, when the patient breathes out. During inhalation, an improved mixture is provided due to the arrangement of the aerosol generator in the air-flow pathway and the arrangement of the aperture surface such that the generated droplets are provided in the inhalation air-flow direction. The by-pass flow path reduces the re-deposition of drugs in form of the small liquids onto the aperture surface of the aerosol generator.

According to an example, the air-flow is manually activatable by the user breathing in and out respectively, and the air-flow is non-supported by ventilation means.

The pure “manual” activation of the air-flow, or in other words the absence of any ventilation means, allows a construction of the nebulizer head with reduced complexity.

According to an example, the air-flow openings are each provided as bi-directional air inlet and air outlet openings.

According to an example, the aerosol generator is arranged within the air-flow path such that by-pass flow paths are arranged around the aerosol generator.

According to an example, one of the air-flow openings is provided as a mouth-piece opening and the other one of the air-flow openings is provided as a backside opening. A flow direction from the backside opening to the mouth-piece opening is the first air-flow direction and the vice versa flow direction is the second air-flow direction.

In an example, the aerosol generator provides the small droplets in the inhaling direction and during inhaling, a flow of air flowing around the aerosol generator is provided at a rear side of the aperture surface to then mix with the droplets at the front side.

According to an example, the backside opening is arranged behind the aperture surface.

According to an example, a portion of the air-flow path between the aerosol generator and the mouth-piece opening is a front air channel and a portion between the aerosol generator and the backside opening is a rear air channel. The rear air channel and preferably the by-pass sections are equal or larger in clear cross-section than the mouth-piece opening to minimize resistance.

According to an example, the rear air channel is angled upward in the exhaling direction.

According to an example, the mouth-piece is provided as a fixedly mounted mouth-piece.

According to an example, the mouth-piece provides the front air channel having a length between the mouth-piece opening and the aperture member, which length is larger than 5 cm.

According to an example, the mouth-piece opening is oval in shape.

According to an example, the aperture member is provided as one of the group of a mesh or membrane with a plurality of holes or apertures, and a plate with a plurality of droplet outlets.

According to the invention, also a nebulizer system is provided with a nebulizer head and a base structure. The nebulizer head is provided as a nebulizer head according to one of the above-mentioned examples. The base structure comprises a power supply and a control and operating element for operating the aerosol generator. The nebulizer head is at least temporarily mounted to the base structure.

According to the present invention, also a method for providing a substance in an aerosolized form using a nebulizer is provided. The method comprises the following steps:
a) In a first step, the substance is provided as aerosol droplets within an air-flow caused by inhalation of the patient. The air-flow is entering an air-flow path at a rear air-flow opening and is flowing around an aerosol generator at least partly such that the aerosol droplets mix with the air-flow to form an aerosol. The rear air-flow opening is facing away from a patient inhaling. The aerosol is exiting the air-flow path at a mouth-piece opening to enter a patient's mouth or nose for further reaching the patient's air passages and respiratory tracts.
b) In a second step, the exhaled air is discharged. The exhaled air is entering the air-flow path at the mouth-piece opening and is flowing around the aerosol generator at least partly such that the aerosol droplets are transported towards the rear air-flow opening. The air mixture is exiting the air-flow path at the rear air-flow opening to be discharged to the surrounding air away from the patient exhaling.

In an example, the substance is a medical drug. In a further example, the substance is a liquid that improves the patient's wellbeing.

According to an aspect, a nebulizer head is provided with an air-flow path and the aerosol generator is arranged within the air-flow path such that due to arranging at least one by-pass flow path section, the aerosol generation is improved in view of the inhalation procedure, and the discharge of the air/droplet mixture is improved in view of the exhalation procedure. The aerosol generator is arranged so to speak within the air-flow path.

These and other aspects of the present invention will become apparent from and be elucidated with reference to the embodiments described hereinafter.

### BRIEF DESCRIPTION OF THE DRAWINGS

Exemplary embodiments of the invention will be described in the following with reference to the following drawings:

FIG. 1A shows a schematic cross-section through a nebulizer head and the resulting air-flow and aerosol generation during inhalation;

FIG. 1B shows a respective schematic illustration of the air-flow during exhalation;

FIG. 2 shows a perspective illustration of the air-flow during inhalation;

FIG. 3 shows a vertical cross-section through the nebulizer head;

FIG. 4 shows a vertical cross-section transverse to the viewing direction of FIG. 3 through a nebulizer head;

FIG. 5 shows a schematic illustration of a nebulizer system; and

FIG. 6 shows a schematic illustration of basic steps of a method for providing a substance in an aerosolized form.

### DETAILED DESCRIPTION OF EMBODIMENTS

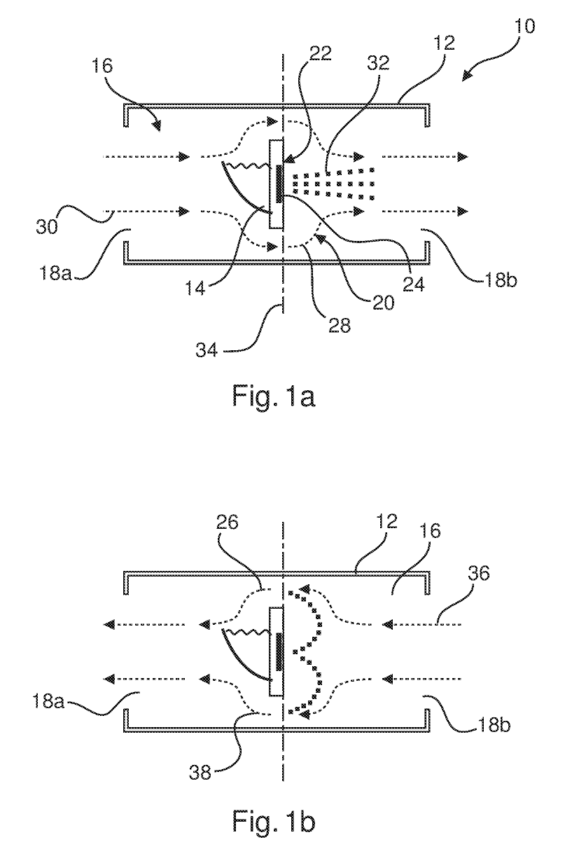

FIG. 1A shows a schematic section through a nebulizer head 10 that comprises a housing structure 12 and a fluid reservoir 14. The fluid reservoir 14 is provided to accommodate a liquid from which small droplets are to be generated in order to form an aerosol. The housing structure 12 provides an air-flow path 16 with two opposite air-flow openings 18a, 18b. Further, an aerosol generator 20 is provided that comprises an aperture member 22. The aperture member 22 is provided with an aperture surface 24 with a plurality of apertures (not further shown) to provide the small droplets from the liquid.

The fluid reservoir 14 is arranged adjacent the aerosol generator 20 such that the aperture member 22 is in contact with the fluid. The aerosol generator 20 is arranged within the air-flow path 16. The fluid reservoir 14 may be accessible from the top, for example via an opening.

At least one by-pass flow path 26 is provided between the aerosol generator 20 and the housing structure 12. The air-flow path 26 is a bi-directional path that provides a first air-flow direction 28 for inhaling. In FIG. 1A, dotted arrows 30 illustrate the resulting air-flow during the inhalation. Further dotted arrows 32 illustrate the generation of the liquid droplets. A hashed line 34 illustrates a plane, in which the aperture surface 24 may be arranged.

FIG. 1B shows the air-flow during exhalation. Dotted arrows 36 illustrate the resulting air-flow. As can be seen, the bi-directional path provides a second air-flow direction 38 for exhaling.

The air enters the air-flow path and leaves the air-flow path via the air-flow openings 18a, 18b. The aperture surface 24 is arranged transverse to the first and second air-flow direction 28, 38.

In an example, a plane, in which the aperture member is arranged, for example the mesh plane 34, is provided perpendicular to the flow direction.

The aerosol generator can also be referred to as aperture member generator.

The first air-flow direction is an inhaling or inhalation direction and the second air-flow direction is an exhaling or exhalation direction.

In an example, the air-flow is manually activatable by the user breathing in and out respectively.

An advantage of the above described arrangement is the generation of the aerosol droplets into the air-flow path having a similar direction in order to achieve a well distributed aerosol to be inhaled by the patient.

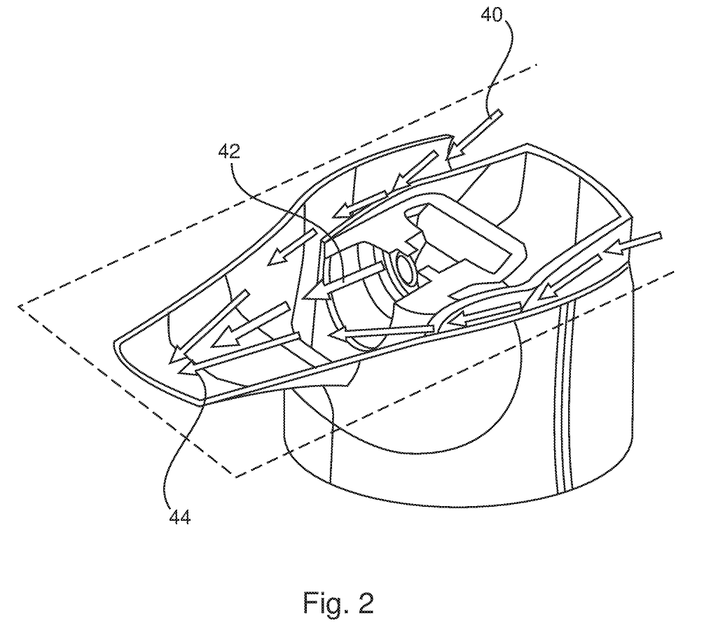

This is also indicated in FIG. 2, in which first arrows 40 indicate an air-flow during inhalation and second arrows 42 indicate the generation of the aerosol droplets to provide an aerosolized drug delivery to the patient at a mouth-piece opening 44.

During exhalation, the by-pass flow path 26 avoids the re-deposition of the aerosol droplets and rather allows a circumvention of the aerosol generators aperture surface, as indicated in FIG. 1B.

Hence, as an example, it is provided that the air-flow openings are each provided as bi-directional air inlet and air outlet openings.

As an option, it is shown to arrange the aerosol generator 20 within the air-flow path 16 such that by-pass flow paths are arranged around the aerosol generator. For example, at least a portion of the perimeter of the aerosol generator is provided with by-pass channels.

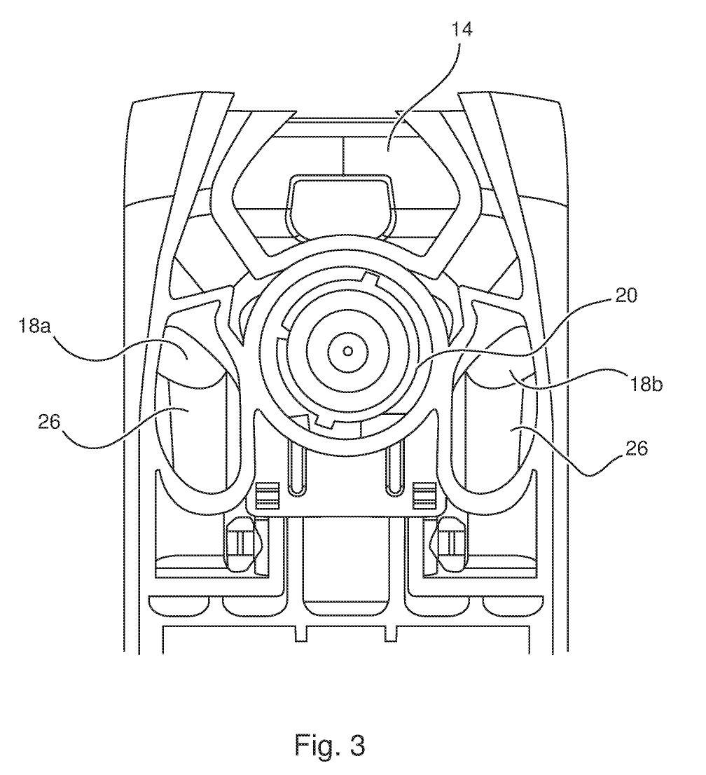

FIG. 3 indicates the aerosol generator 20 and respectively arranged by-pass flow paths 26 on either side.

In another example, not further shown, the aerosol generator 20 is provided with by-pass flow paths all around. In another example, several by-pass flow paths are provided in an approximately symmetrical manner around the aerosol generator.

In an example, the aperture member is mounted substantially vertically in the head. The term “vertically” relates to the normal use when the air-flow path is arranged horizontally.

As a further option, FIG. 3 also indicates the arrangement of the fluid reservoir 14 above the aerosol generator 20, but which fluid reservoir 14 has a downwardly reaching extension such that the liquid can be provided on the respective side of the aerosol generator.

As a further option, the by-pass flow paths 26 are arranged as upward sloping venting ducts towards the rear.

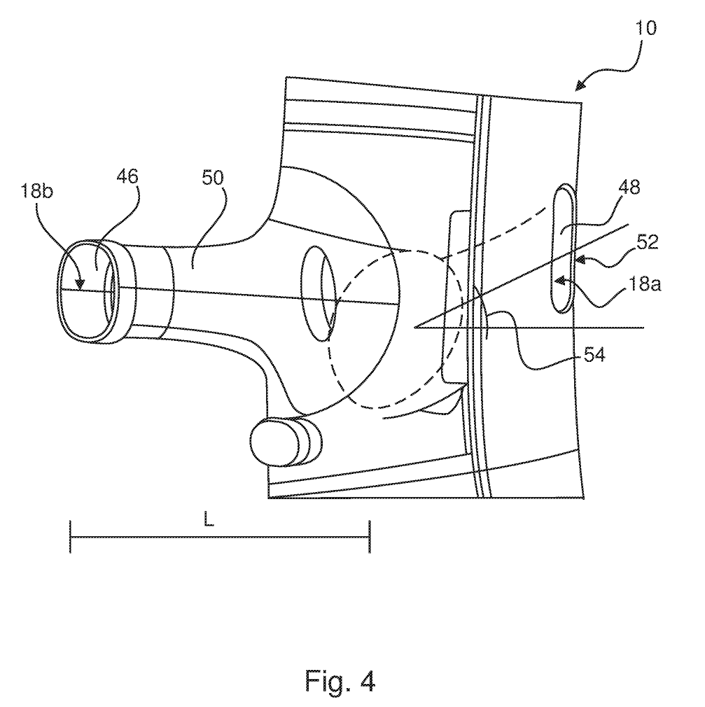

FIG. 4 shows the nebulizer head 10, and as an option the arrangement of one of the air-flow openings being provided as a mouth-piece opening 46, and the other one of the air-flow openings is provided as a backside opening 48. A flow direction from the backside opening 48 to the mouth-piece opening 46 is the first air-flow direction and the vice versa flow direction is the second air-flow direction.

As an option, as already indicated above, the aerosol generator 20 provides the small droplets in the inhaling direction, and during inhaling, a flow of air flowing around the aerosol generator 20 is provided at a rear side of the aperture surface 24.

As an option, the backside opening is arranged behind the aperture surface 24.

The backside opening is arranged downstream the aperture surface in the exhaling air-flow direction, and upstream in the inhaling air-flow direction. The backside opening is arranged at a distal end when the user is breathing through the mouthpiece.

As a further option it is indicated in FIG. 4 that a portion of the air-flow path between the aerosol generator and the mouth-piece opening is a front air channel 50 and a portion between the aerosol generator and the backside opening is a rear air channel 52. The rear air channel 52 and preferably the by-pass sections, i.e. the by-pass flow paths 26, are equal or larger in clear cross-section than the mouth-piece opening. This further improves the air-flow within the nebulizer head.

As a further option it is indicated in FIG. 4 that the rear air channel 52 is angled upward in the exhaling direction, as indicated with angle symbol 54. The air channel is thus angled upwards towards the back side opening. In a normal handling position, the mouth-piece is arranged in a horizontal manner, and the aperture member is hence in a vertical manner.

As a further option, the mouth-piece 46 is provided as a fixedly mounted mouth-piece. The mouth-piece is hence not removable.

As a further option, it is provided that the mouth-piece 46 provides the front air channel 50 having a length L between the mouth-piece opening and the aperture member, which length L is larger than 5 cm. The mouth-piece opening may be oval in shape.

The aperture member 22 may be provided as a mesh or membrane with a plurality of holes or apertures. The aperture member 22 may also be provided as a plate with a plurality of droplet outlets.

The configuration of the exit outlet or conduit in an angled manner away from the mesh surface prevents direct inline access to the mesh surface and the length is increased to ensure a finger cannot come into contact with the mesh surface. Preferably, this angle between an horizontal axis and a longitudinal axis of the mouthpiece may be greater than five degrees.

In an example, the exit conduit is angled away from the center line of the mesh and combined with the increased length, and the exit port at the mouth-piece is oval in shape. This geometry restricts the patient from accessing the mesh. The conduit may be angled greater than 5° and lengthened to a minimum of 5 cm with a minimum cord of 2 cm for the outlet oval shape.

The arrangement of the above-mentioned clear cross-section relations allows to provide a low pressure resistance to tidal breathing flow rates.

To arrange the rear airway channel angled upward also improves the situation due to dipping from condensate when the aerosol impacts the inner walls of the air channel, since the slope allows for the condensate to be captured during treatment and may then be disposed off after treatment. As an added benefit, the rear vents are positioned to minimize the action of a patient from covering the vents when holding the device.

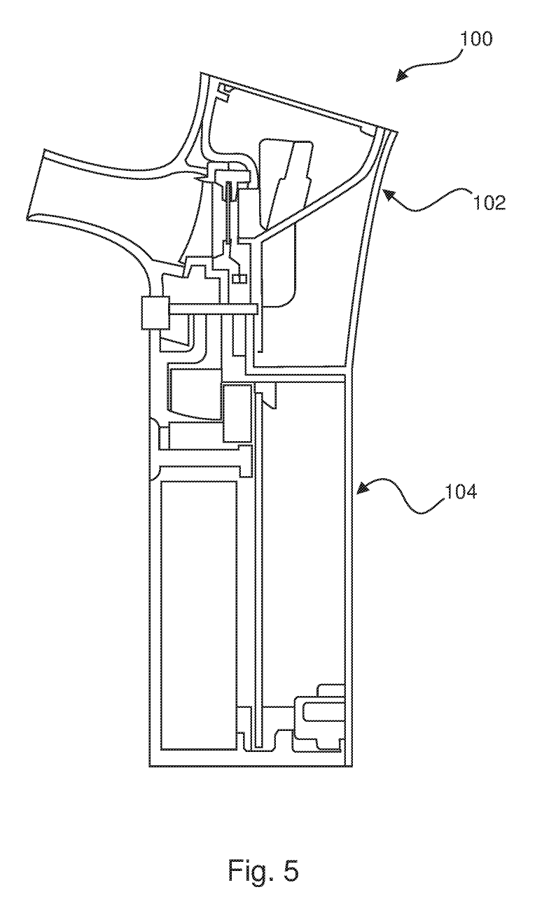

In FIG. 5, a nebulizer system 100 is illustrated in a schematic cross-section. The nebulizer system 100 comprises a nebulizer head 102 and a base structure 104. The nebulizer head 102 is provided as one of the above-mentioned examples of the nebulizer head 10. The base structure comprises a power supply and a control and operating element for operating the aerosol generator. The nebulizer head 102 is at least temporarily mounted to the base structure.

As indicated in FIG. 5, the nebulizer head is provided for a hand-held device. However, the nebulizer head may also be provided for a portable device, in which the base structure 104 would not form a hand-held grip portion, but would be a base station.

As an option, the nebulizer head may be mounted removably.

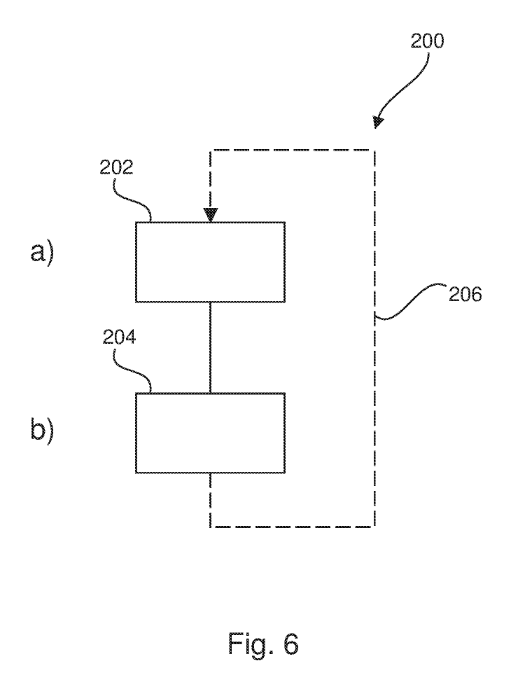

FIG. 6 shows a method 200 for providing a substance in an aerosolized form using a nebulizer. The method 200 comprises the following steps: In a first step 202, also referred to as step a), the substance is provided as aerosol droplets within an air-flow caused by inhalation of the patient. The air-flow is entering an air-flow path at a rear air-flow opening and is flowing around an aerosol generator at least partly such that the aerosol droplets mix with the air-flow to form an aerosol. The rear air-flow opening is facing away from a patient inhaling. The aerosol is exiting the air-flow path at a mouth-piece opening to enter a patient's mouth or nose for further reaching patient's air passages and respiratory tracts. In a second step 204, also referred to as step b), the exhaled air is discharged. The exhaled air is entering the air-flow path at the mouth-piece opening and is flowing around the aerosol generator at least partly such that the aerosol droplets are transported towards the rear air-flow opening. The air mixture is exiting the air-flow path at the rear air-flow opening to be discharged to the surrounding air away from the patient exhaling.

The first and second steps 202, 204 may then be repeated, as indicated with repeat loop arrow 206.

It has to be noted that embodiments of the invention are described with reference to different subject matters. In particular, some embodiments are described with reference to an nebulizer head whereas other embodiments are described with reference to the system and the method. However, a person skilled in the art will gather from the above that, unless otherwise notified, in addition to any combination of features belonging to one subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfil the functions of several items re-cited in the claims. The mere fact that certain measures are re-cited in mutually different dependent claims does not indicate that a combination of these measures cannot be used to advantage. Any reference signs in the claims should not be construed as limiting the scope.

1. A nebulizer head, comprising:
a housing structure providing an air-flow path with two opposite air-flow openings;
a fluid reservoir provided to accommodate a liquid from which small droplets are to be generated in order to form an aerosol;
an aerosol generator with an aperture member that comprises an aperture surface with a plurality of apertures to provide the small droplets from the liquid; wherein the fluid reservoir is arranged adjacent the aerosol generator such that the aperture member is in contact with the fluid;
wherein the aerosol generator is arranged within the air-flow path;
wherein at least one by-pass flow path is provided between the aerosol generator and the housing structure;
wherein the air-flow path is a bidirectional path that provides a first air-flow direction for inhaling, and a second air-flow direction for exhaling;
wherein the air enters the air-flow path and leaves the air-flow path via the air-flow openings; and
wherein the aperture surface is arranged transverse to the first and second air-flow direction.

2. Nebulizer head according to claim 1, wherein the air-flow is manually activatable by the user breathing in and out respectively; and wherein the air-flow is non-supported by ventilation means.

3. Nebulizer head according to claim 1, wherein the air-flow openings are each provided as bi-directional air inlet and air outlet openings.

4. Nebulizer head according to claim 1, wherein the aerosol generator is arranged within the air-flow path such that by-pass flow paths are arranged around the aerosol generator.

5. Nebulizer head according to claim 1, wherein one of the air-flow openings is provided as a mouth-piece opening and the other one of the air-flow openings is provided as a backside opening; and a flow direction from the backside opening to the mouth-piece opening is the first air-flow direction and the vice versa flow direction is the second air-flow direction; and wherein, preferably, the aerosol generator provides the small droplets in the inhaling direction; and wherein during inhaling a flow of air flowing around the aerosol generator is provided at a rear side of the aperture surface.

6. Nebulizer head according to claim 1, wherein the backside opening is arranged behind the aperture surface.

7. Nebulizer head according to claim 5, wherein a portion of the air-flow path between the aerosol generator and the mouth-piece opening is a front air channel and a portion between the aerosol generator and the backside opening is a rear air channel; and wherein the rear air channel and preferably the by-pass sections are equal or larger in clear cross-section than the mouth-piece opening.

8. Nebulizer head according to claim 1, wherein the rear air channel is angled upward in the exhaling direction.

9. Nebulizer head according to claim 7, wherein the mouth-piece is provided as a fixedly mounted mouth-piece.

10. Nebulizer head according to claim 5, wherein the mouth-piece provides the front air channel having a length (L) between the mouth-piece opening and the aperture member, which length is larger than 5 cm; and wherein, preferably, the mouth-piece opening is oval in shape.

11. Nebulizer head according to claim 5, wherein the longitudinal axis of the mouthpiece makes an angle greater than five degrees with respect to an horizontal axis.

12. Nebulizer head according to claim 1, wherein the aperture member is provided as one of the group of a mesh or membrane with a plurality of holes or apertures, and a plate with a plurality of droplet outlets.

13. A nebulizer system comprising:
a nebulizer head comprising:
a housing structure providing an air-flow path with two opposite air-flow openings;
a fluid reservoir provided to accommodate a liquid from which small droplets are to be generated in order to form an aerosol;
an aerosol generator with an aperture member that comprises an aperture surface with a plurality of apertures to provide the small droplets from the liquid, wherein the fluid reservoir is arranged adjacent the aerosol generator such that the aperture member is in contact with the fluid, wherein the aerosol generator is arranged within the air-flow path wherein at least one by-pass flow path is provided between the aerosol generator and the housing structure, wherein the air-flow path is a bidirectional path that provides a first air-flow direction for inhaling, and a second air flow direction for exhaling, wherein the air enters the air-flow path and leaves the air-flow path via the air-flow openings, and wherein the aperture surface is arranged transverse to the first and second air-flow direction; and
a base structure comprising a power supply and a control and operating element for operating the aerosol generator; wherein the nebulizer head is at least temporarily mounted to the base structure.

14. A method for providing a substance in an aerosolized form using a nebulizer, the method comprising the following steps:
a) providing the substance as aerosol droplets within an air-flow caused by inhalation of the patient; wherein the air-flow is entering an air-flow path at a rear air-flow opening and is flowing around an aerosol generator at least partly such that the aerosol droplets mix with the air-flow to form an aerosol; wherein the rear air-flow opening is facing away from a patient inhaling; and wherein the aerosol is exiting the air-flow path at a mouth-piece opening to enter a patient's mouth or nose for further reaching patient's air passages and respiratory tracts; and
b) discharging (204) the exhaled air; wherein the exhaled air is entering the air-flow path at the mouth-piece opening and is flowing around the aerosol generator at least partly such that the aerosol droplets are transported towards the rear air-flow opening; and wherein the air mixture is exiting the air-flow path at the rear air-flow opening to be discharged to the surrounding air away from the patient exhaling.
